(19) United States
(12) Patent Application Publication
(10) Pub. No.: US 2025/0213832 A1
(43) Pub. Date: Jul. 3, 2025

(54) CATHETER

(71) Applicant: SRN Medical Devices Ltd, Felden Hertfordshire (GB)

(72) Inventors: Colin J. Nicholl, Horsham (GB); Jonathan Ramsay, Henley on Thames (GB); David BA Silk, King's Lynn (GB)

(73) Assignee: SRN Medical Devices Ltd, Felden Hertfordshire (GB)

(21) Appl. No.: 18/852,397

(22) PCT Filed: Mar. 31, 2023
(86) PCT No.: PCT/GB2023/050861

§ 371 Date: Sep. 27, 2024

(30) Foreign Application Priority Data
Mar. 31, 2022 (GB) 2204656.9

(51) Int. Cl. A61M 27/00; A61J 15/00; A61M 3/02

(52) U.S. Cl. CPC A61M 27/008; A61J 15/0069; A61J 15/0073; A61M 3/0283; A61M 2210/1089

(57) ABSTRACT

A urethral catheter comprising an elongate generally cylindrical body and a catheter tip. The catheter comprises an irrigation lumen, a drainage lumen and optionally an inflation lumen in fluid communication with an expandable member. The catheter is arranged to communicate fluid to and from the catheter tip.

### BACKGROUND

The present invention is concerned with alternative catheter arrangements for use in a variety of medical applications. Specifically, but not exclusively, the catheters described herein may be used in urethral catheterisation, surgical drainage and in stomach decompression tubes and enteral (gastric and small intestine) feeding tubes.

An example of the use of catheters is where patients' micturition (urination) can be problematic. We consider briefly the use of urethral catheters as one example of the importance of catheters in general. Their use may be for various reasons including patient age, patients being infirm or because of patients suffering post-operative complications. In such situations the patient may have lost, or have limited ability to control micturition (passage of urine). Some male patients may have simple obstruction to the bladder, caused by enlargement of the prostate and may be too ill or frail to undergo surgery.

In general, the majority of patients requiring urethral catheterisation will have some failure of the function of the bladder or of its control mechanisms and the procedure is of increasing relevance as the population in the western world ages. The bladder is a muscular organ and, as the muscle of this organ weakens with age, the ability of the bladder to contract and thus empty satisfactorily, is lost. A small number of patients are incontinent due to the muscular valves that control micturition ceasing to function and bladder catheterisation is the treatment of last resort. Elderly demented patients may also present with incontinence and this may be due to lack of cognitive function rather than dysfunction of the lower urinary tract but all require urethral catheterisation.

Current solutions to these situations may include the insertion along the urinary passage into the bladder of either a temporary intermittent catheter or an in-dwelling catheter that will remain in place for a number of weeks. The bladder can then be drained through the catheter and out of the body. These solutions are simple to use and are reliable by virtue of the simplicity of design.

However, although simple to use and reliable in operation, existing urethral catheters present their own drawbacks and complications. These include blockages and bladder inflammation with the latter usually associated with Catheter Associated Urinary Tract Infection. (CAUTI).

Blockages occur for a number of reasons. For example, the lining of the bladder can occlude the drainage port in the catheter dependent on its position relative to the wall within the bladder. Another complication occurs when the bladder wall becomes irritated by the presence of the catheter and the response of the glands in the bladder wall is to become inflamed and produce mucus which then blocks the drainage port of the urethral catheter. This is particularly the case for in-dwelling catheters i.e. catheters that are in-situ for longer periods of time. Separately, blockages within the bladder can be caused by post-operative blood clots resulting from prostate surgery or tumour surgery in the bladder that has resulted in a raw area that bleeds.

In summary, although existing urethral catheters are simple to use, they do present complications that frequently require the catheter to be removed and replaced. Disadvantageously this thereby consumes important clinician time, causes distress to the patient further increases the risk of CAUTI, and importantly increases overall costs.

The Applicant has devised an alternative approach to catheterisation and specifically a catheter design that advantageously addresses a number of the drawbacks of existing catheter technology. Furthermore, the Applicant has devised a design that can advantageously be used not just to address complications with urethral catheters but also to address complications with surgical drainage, gastric decompression and naso-gastric and naso-intestinal enteral feeding. The novel and inventive approach to catheter design is not limited to catheters for human use; the approach may equally be applied in these areas for veterinary procedures.

### SUMMARY OF THE INVENTION

Aspects of the invention are set out in the accompanying claims.

Viewed from a first aspect of an invention described herein there is provided, a catheter comprising an elongate generally cylindrical body and a catheter tip for insertion into a human or animal, the elongate generally cylindrical body comprising a first fluid ingress lumen and a second fluid egress lumen for communicating fluid along the elongate generally cylindrical body to and from the catheter tip, wherein the catheter tip comprises a first portion integral with the elongate generally cylindrical body, a second portion distal to the first portion and defining the distal end of the catheter and an intermediate fluid ingress/egress region located between the first and second portions, wherein the intermediate fluid ingress/egress region is in the form of an open section of the elongate cylindrical body exposing the first and second lumens to allow for fluid communication; the catheter tip further comprising a base portion extending between the first and second catheter tip portions; and wherein the fluid ingress lumen extends through a predetermined length of the base portion and is arranged in use to discharge fluid from an outlet port to direct fluid away from the base portion and into the intermediate fluid ingress/egress region.

Thus, an alternative catheter geometry is provided that causes fluid to flow into and out of the bladder in such a way that blockages or occlusion of outlet passages can be avoided. Further advantages are described herein.

Specifically the open nature of the intermediate ingress/egress region (also called the aspiration port) provides a volume or zone in which irrigation fluid can mix with surrounding urine and pass to the egress port. Advantageously, the way in which the outlet port is geometrically configured prevents a mucosa or similar from coming into close contact with the catheter proximate to an outlet and blocking the catheter in use.

The intermediate fluid ingress/egress region may be in the form of a generally concave recess in the cylindrical body extending towards the base, the recess defining an upper opening relative to the base which intersects with the lumens to allow fluid flow to and from the lumens. Such a recess may be conveniently moulded for example in a material suitable for use in a body.

In such an arrangement the recess may advantageously intersect with the base and first and second catheter tip portions to define a smooth and continuous surface profile. This facilitates smooth fluid flow and prevents trauma caused by the catheter contracting the muscosa or vein wall, for example. It also prevents fluid turbulence.

At least a portion of the intermediate fluid ingress/egress region may extend more than 180 degrees around the circumference of the cylindrical body. Such an angle advantageously provides sufficient structural material strength to the distal end of the catheter whilst providing the recess described above and herein. For example, at least a portion of the intermediate fluid ingress/egress region may extend between 180 degrees and 190 degrees around the circumference of the cylindrical body.

The fluid egress lumen may be coaxial with the elongate generally cylindrical body and intersect with the intermediate fluid ingress/egress region to allow fluid flow therefrom. Such a configuration facilitates fluid flow, allows the geometry to be conveniently manufactured and allows a minimal overall diameter of catheter to be achieved. Alternatively it allows a large cross-section of lumen for a given cross-sectional area.

The fluid ingress lumen may extend along the elongate generally cylindrical body and intersect with the intermediate fluid ingress/egress region from the base portion to allow fluid flow substantially perpendicularly therefrom. Thus, fluid entering the intermediate region or zone has a component of movement away from the catheter in a direction that advantageously intersects with tissue, such as mucosa or vein walls that may be proximate thereto. Thus, a hydraulic pressure and thus force can be applied to maintain the space between the mucosa and the ingress and egress ports. Fluid may advantageously be arranged to flow from an outlet of the fluid ingress lumen so as to flow away from the inlet to the egress lumen.

Viewed in cross-section, the intermediate fluid ingress/egress region may advantageously have a smooth generally U shaped, ‘bowl’ shape or ‘concave’ profile. Thus a space or void can be created by the fluid flow, allowing urine or other fluid to be drained to pass towards the egress port.

The second portion of the catheter tip defining the distal end of the catheter may also have a smooth generally curved outer surface. In effect, a ‘bullet’ or ‘convex’ geometry may be provided. Such a surface profile advantageously allows the catheter to be inserted along the urethra or vein with minimal discomfort or trauma to the patient and furthermore advantageously allows for smooth fluid flow around the catheter tip once in situ in the body.

The second portion of the catheter tip defining the distal end of the catheter may be inclined with respect to the elongate axis of the catheter. This may, for example, be inclined at an angle of between 15 degrees and 30 degrees. Such an inclination allows the catheter to be navigated or inserted through the urethra with minimal trauma and pain to the patient. For example, the second portion may be inclined at an angle of approximately 15 degrees.

The catheter may also advantageously comprise an expandable member located circumferentially with respect to the elongate generally cylindrical body and movable radially outwards from the body surface. Such an expandable member allows the catheter (a) to be inserted with the member in an un-expanded state, thereby minimising the diameter of the catheter and (b) provides a mechanism to selectively secure the catheter in place by expansion of the member at an appropriate position and to an appropriate diameter.

In one example, the expandable member may be in the form of a circumferentially located balloon expandable between a first position in alignment with the outer surface of the elongate body and a second position radially spaced from the body. A balloon conveniently allows a light and simple expandable member to be provided that may be expanded with a fluid such as water or saline solution.

The catheter may also comprise a lumen extending along the elongate body and in fluid communication with the expandable member to cause selective expansion or retraction of the expandable member. Thus the member can be accurately controlled.

The elongate, generally cylindrical body, may comprise a first lumen for fluid egress and two radially opposing lumens, a first being a fluid ingress lumen and a second being in fluid communication with an expandable member. The expandable member may be in the form of a balloon which may be selectively expanded by means of a fluid.

Furthermore, the expandable member may be positioned at least 4 cm from the tip of the catheter to the midpoint of the expandable member inflation channel i.e. a midpoint at which the balloon is inflated.

For a surgical drain, the expandable member (for example balloon) may be positioned 10, 20 or 30 cm from the tip of the catheter to the midpoint of the expandable (for example balloon) inflation channel i.e a midpoint at which the balloon is inflated from the channel.

The catheter may be formed from any suitable material. However the catheter may advantageously be formed from a material selected from an aromatic polyurethane teflon, polyvinylchloride (PVC) and silicone. Such materials are soft enough to prevent trauma in a patient, convenient to manufacture and provide sufficient structure strength for the application.

They are also tolerated by the human body. Polyurethane is particularly advantageous since or any given outer diameter of catheter the greatest internal diameter of lumen can be achieved whilst maintaining sufficient structural strength of the catheter. Furthermore, it is also more biocompatible with the human body than other materials.

For example, the catheter may be formed from a polyurethane material having a Shore Hardness of between 85 and 90 Shore A.

The maximum length of the intermediate fluid ingress/egress region extending between the first portion of the catheter tip and the second portion of the catheter tip when measured along the axis of the catheter may be less than 15 mm.

Viewed from a further aspect there is provided a catheter as described herein wherein the catheter is a urethral catheter or surgical drainage catheter.

Viewed from yet another aspect there is provided a urethral catheter comprising an elongate generally cylindrical body and a catheter tip for insertion into and along a urethra,
the elongate generally cylindrical body comprising:
an irrigation lumen for communicating fluid to the catheter tip,
a drainage lumen for receiving fluid from the catheter tip, and
optionally an inflation lumen in fluid communication with an expandable member;
wherein the catheter tip has a first and second mucosa supporting portion and an intermediate opening between the first and second mucosa supporting portions, wherein the opening extends between 180 degrees and 190 degrees around the circumference of the elongate generally cylindrical body, the intermediate opening being in fluid communication with the irrigation lumen and the drainage lumen.

Viewed from a still further aspect there if provided a method of urethral or surgical drainage or gastric decompression or enteral feeding tube for use in the stomach and small intestine by means of a catheter as claimed in any preceding claim, the method comprising the steps of inserting a catheter into a body, causing fluid to ingress into a body and removing fluid from the body through the fluid egress lumen

Aspects of catheters described herein may also be used in peripheral venous catheters and central venous catheters for delivery of intravenous fluids, blood drugs and parenteral nutrition and in addition to irrigation and drainage of fluids.

The outside diameter of the catheters may vary depending on application but in general will be between 5.3 mm and 6.6 mm (16-20 Fr) for urethral catheters with a maximum internal diameter between 3.9 mm and 5.2 mm. Venous catheters will have a smaller outer diameter, typically less than 16 Fr.

### DRAWINGS

Aspects of the invention will now be described, by way of example only, with reference to the accompanying figures in which:

FIG. 1 shows a schematic of a typical bladder catheter drainage;

FIG. 2 shows the positioning of a catheter in a male patient;

FIG. 3A, 3B and 3C show 3 stages of catheter usage with bladder drainage and contraction;

FIG. 4 shows a conventional catheter tip with an aperture passing through the catheter;

FIG. 5A shows a mucosa and walls internal to other areas of the body occluding the drainage port of conventional catheters and FIG. 5B shows a cross-section of an alternative geometry of catheter as described herein;

FIG. 6 shows an alternative catheter geometry as described herein for use in venous applications;

FIGS. 7 and 7A to 7C show cross-sections of a urethral catheter described herein;

FIG. 8 shows cross-sections through example urethral catheters;

FIGS. 9, and 10 show further details of a catheter tip according to a urethral catheter described herein;

FIGS. 11A to 11D illustrate different positions of the septum with respect to the zone where the ingress and egress fluids mix;

FIG. 12 illustrates a further embodiment of the distal end of the septum with a fluid directing projections;

FIG. 13 illustrates a still further embodiment of the distal end of the septum with a modified fluid path projection; and

FIGS. 14A to 14C illustrate a plan view of the ingress and egress lumens.

While an invention described herein is susceptible to various modifications and alternative forms, specific embodiments are shown by way of example in the drawings and are herein described in detail. It should be understood however that drawings and detailed description attached hereto are not intended to limit the invention to the particular form disclosed but rather the invention is to cover all modifications, equivalents and alternatives falling within the spirit and scope of the claimed invention.

It will be recognised that the features of the aspects of the invention(s) described herein can conveniently and interchangeably be used in any suitable combination. As stated earlier, it will be recognised that the invention covers not only individual embodiments but also combinations of the embodiments that have been discussed herein.

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to”.

The invention is further described with reference to the following examples. It will be appreciated that the invention as claimed is not intended to be limited in any way by these examples.

### DETAILED DESCRIPTION

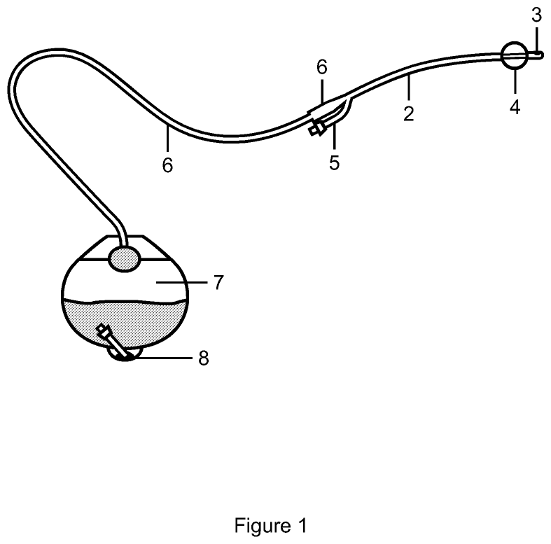

FIG. 1 shows the basic components of a catheter apparatus. In this example, a urethral catheter is shown but, as described herein, aspects of inventions can be used with other catheters and medical drainage and infusion apparatuses.

A catheter apparatus comprises the catheter 2 itself, which is the component which is inserted along the urethra of the patient. The distal end of the catheter 2 comprises a small aperture 3 through which urine may pass from the patient's bladder. The urethral catheter includes balloon 4 which may be inflated with inflation line 5 to maintain the position of the catheter inside the bladder. Once inserted, the catheter 2 is then coupled to the drainage tube 6 which is in turn connected to a collection bag 7 (with optional outlet port 8).

In use, the catheter is inserted and connected to the bag and urine can be drained from the patient's bladder.

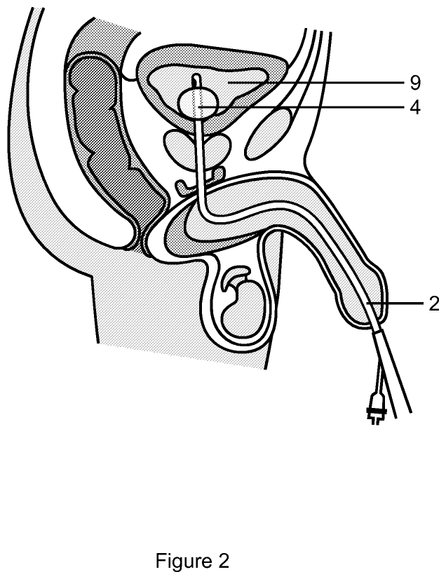

FIG. 2 illustrates the location of an installed catheter 2 in a male patient. As shown, the balloon 4 is inflated and prevents the catheter leaving the bladder 9.

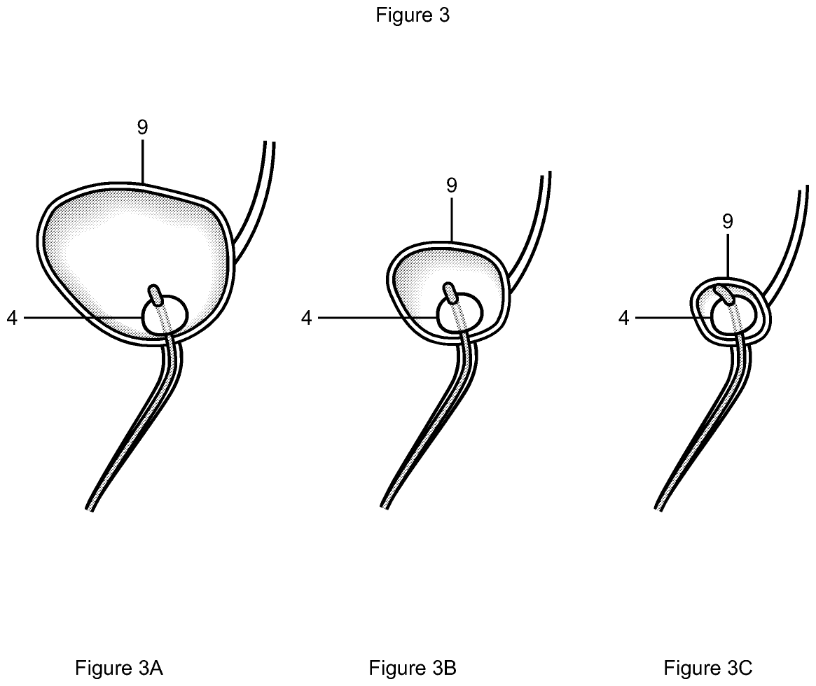

FIGS. 3A to 3C illustrate broadly how the bladder shrinks as urine leaves the bladder. As shown, the bladder closes in around the catheter tip whilst being retained in position by the balloon.

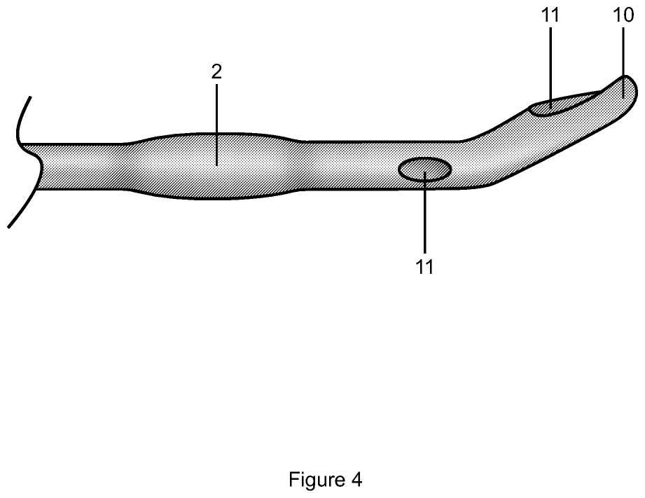

FIG. 4 illustrates a conventional tip of a catheter 2 as commonly used to drain urine from a bladder. As shown, the tip 10 of the catheter 2 comprises an opening 11 which allows urine to pass into the central drainage channel and into a collection bag.

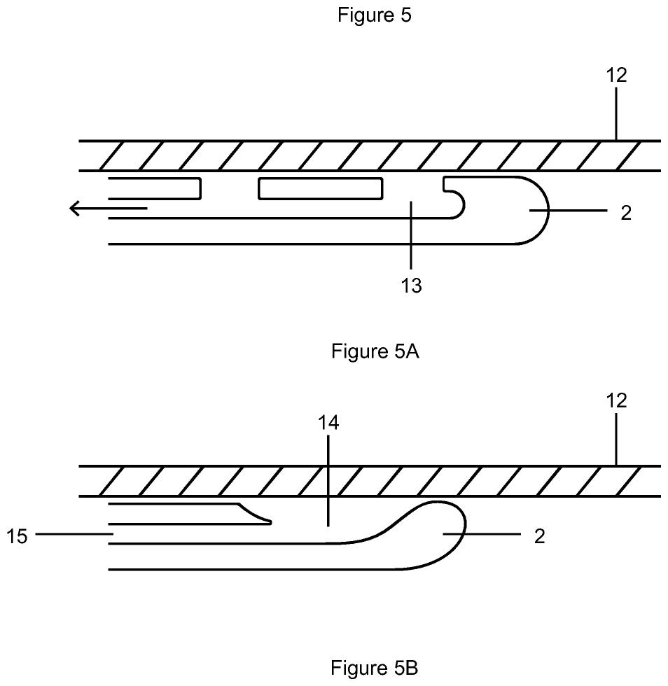

Referring back to FIGS. 3A to 3C, this type of conventional catheter operates perfectly well when the distal end of the catheter is within the middle of the volume of the bladder. However, as the bladder continues to deflate (as urine is removed) the surface of the bladder—the mucosa—eventually comes into contact with the catheter. This is illustrated by FIG. 5A where the mucosa 12 finally abuts with the surface of the conventional catheter 2.

The applicant has established that the majority of medical tubes/catheters have, as their outflow/aspiration port(s), punched outside ports that present a narrow flat surface typically approximating to less than 50-75° of the circumference of the outer diameter (OD) of the tube/catheter (as illustrated in FIG. 4).

As illustrated by FIG. 5A, this type of port 13 can be easily occluded by mucosa 12 (lining) or by the lining of the bladder in the case of urethral catheters, and by mucosa or by intra-abdominal contents (in the case of surgical drains) or the lining of the stomach or small intestine in the case of nasogastric tubes and naso-enteral feeding tubes. In the case of urethral catheters, the tendency to blockage is also exacerbated by blood clots and mucosa plugs that arise when the mucosal surfaces of the bladder are inflamed. In these cases, when an existing tube design is used, once occluded, the flow of urine from the bladder may stop and the catheter either has to be successfully irrigated or removed or moved within the bladder causing discomfort pain trauma and distress to the patient.

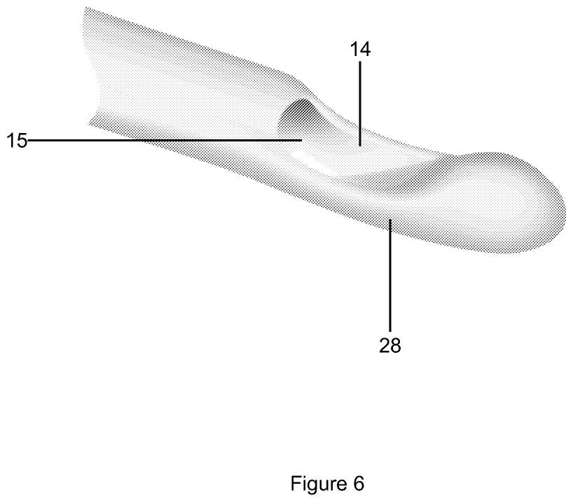

Conversely, FIG. 5B and FIG. 6 shows how the distal end of the catheter according to an invention described herein incorporates a long, wide, smooth and curved opening 14 with no dead space close to the drainage port to trap mucus or blood clots in the case of urethral catheters, or surgical drains and curdled dietary contents in the case of gastric and small intestinal enteral feeding tubes.

FIG. 6 also shows an alternative arrangement in which the novel geometry and configuration of a catheter described herein may be used for intravenous fluid, blood and drug delivery. As described with reference to the bladder mucosa in a urethral application, the same principle may be applied in a vein when the vein wall can be prevented from blocking the catheter by virtue of the geometry of a catheter tip as described herein.

Furthermore, the design of the outflow port is such that the outflow fluid characteristics are novel by virtue of being diffuse rather than concentrated from the tip of existing venous catheters, the “fire-hose effect” (a term in the art) which when aimed at the inside of the lining of the vein causing trauma and inflammation that leads to the development of thrombophlebitis and venous thrombosis, complications that limit the use of peripheral and central vein catheters. Thus, further arrangements described herein provided an improved venous catheter arrangement.

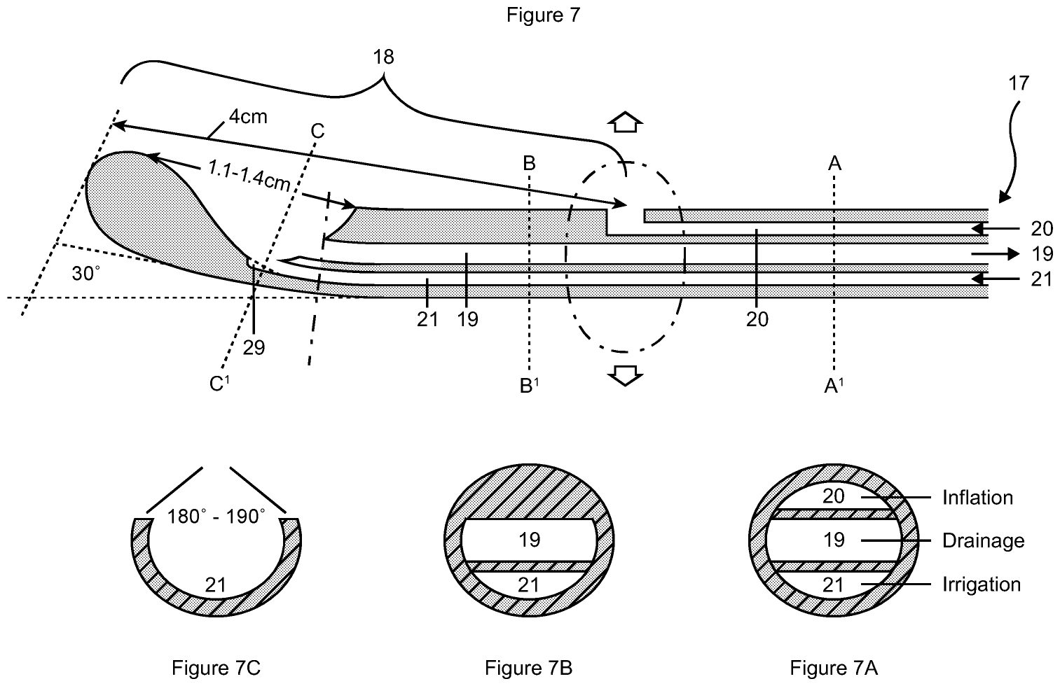

FIGS. 7 and 7A to 7C show further details of catheter design including cross-sectional details of the catheter arrangement.

FIG. 7 shows a urethral catheter example of an invention described herein in cross-section.

Importantly, as shown in FIG. 7, the fluid ingress and egress region extends around the sides of the main catheter body, such that both the top and the sides of the catheter are open to facilitate and increase the fluid flow from the bladder or gastric or intestinal tracts into the egress lumen (this is described further). The impact of this design is to increase the flow of fluid from the body and reduce the frequency of blockages requiring the removal of the catheter compared to conventional tubes. In the case of urethral catheters, it will reduce the volume of urine within the bladder and thereby reduce the incidence of CAUTI.

In the example shown in FIG. 7 a single egress lumen 19 is shown and it will be appreciated that fluid can thus flow from the top and simultaneously from the sides of the catheter and into the lumen.

Cross-sectional views 7A, 7B and 7C illustrate cross-sectional views through the uretheral catheter at the identified points (A-A′; B-B′ and C-C′ respectively). This catheter comprises 3 lumens passing along the elongate body of the catheter. The purpose of each lumen will be described below.

The uretheral catheter comprises a proximal end 17 which is in the form of a generally cylindrical elongate body. At the opposing end is the catheter tip 18.

FIG. 7A shows a cross-section through A-A′ in FIG. 7. As shown 3 lumen are provided in this embodiment but it will be recognised that more or less lumens may be used.

The arrangement in FIG. 7 comprises a first drainage lumen 19 concentric with the elongate body of the catheter and extending from the proximal end (where it is joined to a drainage bag) to the tip 18. The walls of the elongate body 17 also comprise a pair of diametrically opposing lumens 20, 21 (see FIG. 7A). The 3 lumens may be conveniently extruded in order to manufacture the catheter with the 3 lumens integrated into the structure.

Lumen 20 extends along the elongate body and fluidly connects with a balloon 22 arrangement forming a radially expandable element of the embodiment. Introducing a fluid along the lumen 20 causes the balloon to expand as illustrated in FIG. 7 by the dashed and dotted line. The balloon then functions in the same way as a conventional balloon as shown in FIGS. 2 and 3 to 3C to locate the catheter in the body. Removing the fluid by reversing the flow along the lumen 20 will return the balloon to an un-inflated state. Thus, selective control of the expandable member is provided through the lumen 20. The dashed and dotted lines in FIG. 7 illustrate the expansion of a balloon around the circumference of the catheter on application of fluid pressure to lumen 20.

FIG. 7B illustrates a cross-section through B-B′.

In each of FIGS. 7A and 7B the second opposing lumen 21 is also shown, again formed by an extrusion process. Lumen 21 is arranged to communicate an irrigation fluid from the proximal end of the elongate body to the tip 18 (described in more detail below).

FIG. 7A also illustrates the 3 lumens of the catheter:
(i) the upper inflation lumen which acts to communicate fluid to and from the expandable member, typically an elastomeric balloon;
(ii) the drainage lumen which communicates urine and fluid from the distal end; and
(iii) the irrigation lumen which communicates irrigation fluid to the distal end.

The catheter cross-section may be conveniently manufactured using an extrusion process. This allows for a range of cross-sections to be created. The cross-sections shown in FIGS. 7A and 7B are just examples. In another example the irrigation and inflation lumens could be cylindrical lumens formed in the catheter material. It will be recognised that any configuration of lumen size/shape and position may be realised using an extrusion process.

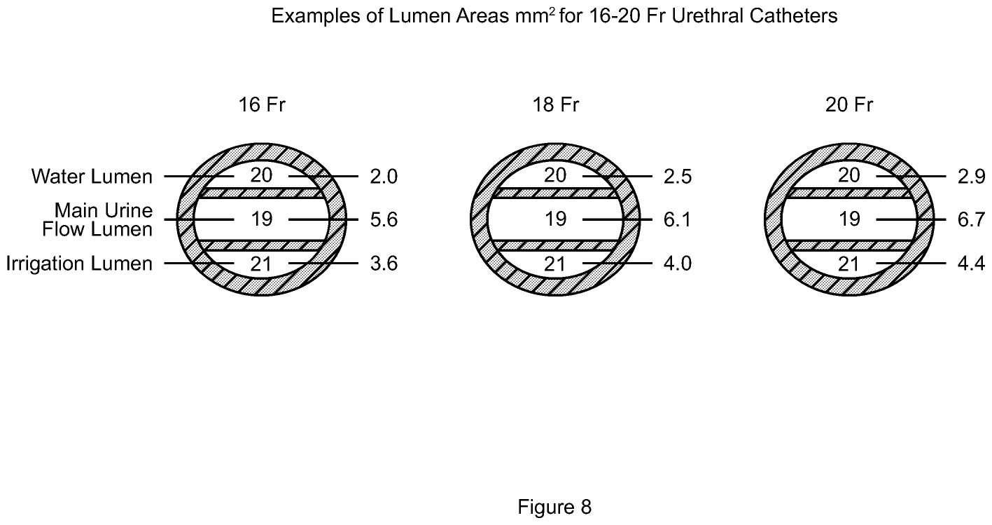

In the examples shown in FIGS. 7A and FIG. 8, instead of 3 circular lumens the cross-section of the elongate body has been segmented, as shown, into 3 passages extending along the elongate body.

FIG. 7C illustrates the cross-section through C-C′ in FIG. 7, i.e. the fluid ingress and egress region at the end of the catheter. It will be recognised that the cross-section shown in FIG. 7 is through the centre of the catheter. The cross-section shown in FIG. 7C illustrates how the sides of the catheter extend around the sides of the catheter corresponding to the outer cylindrical profile.

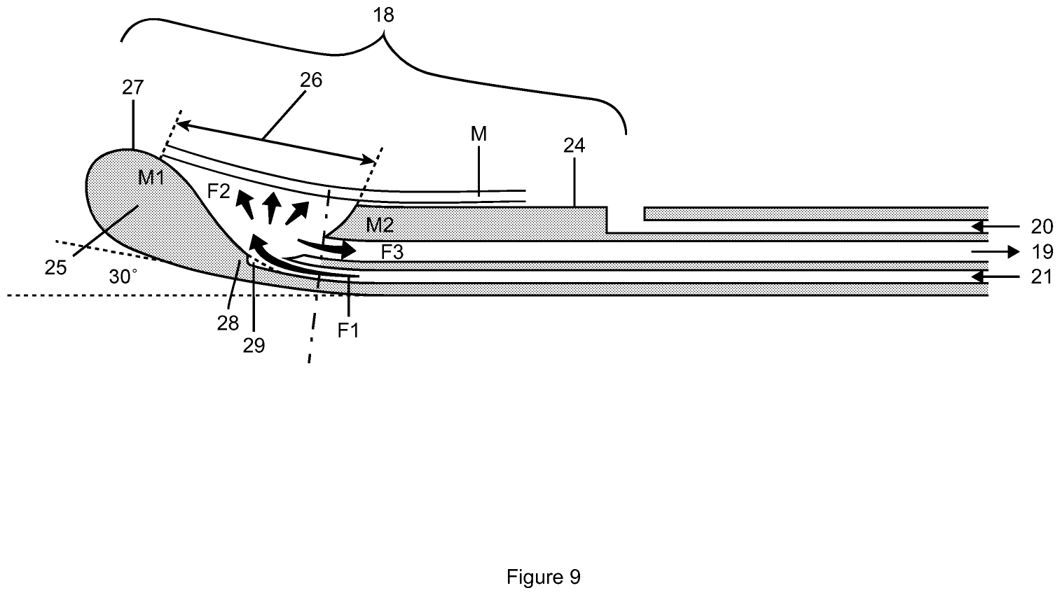

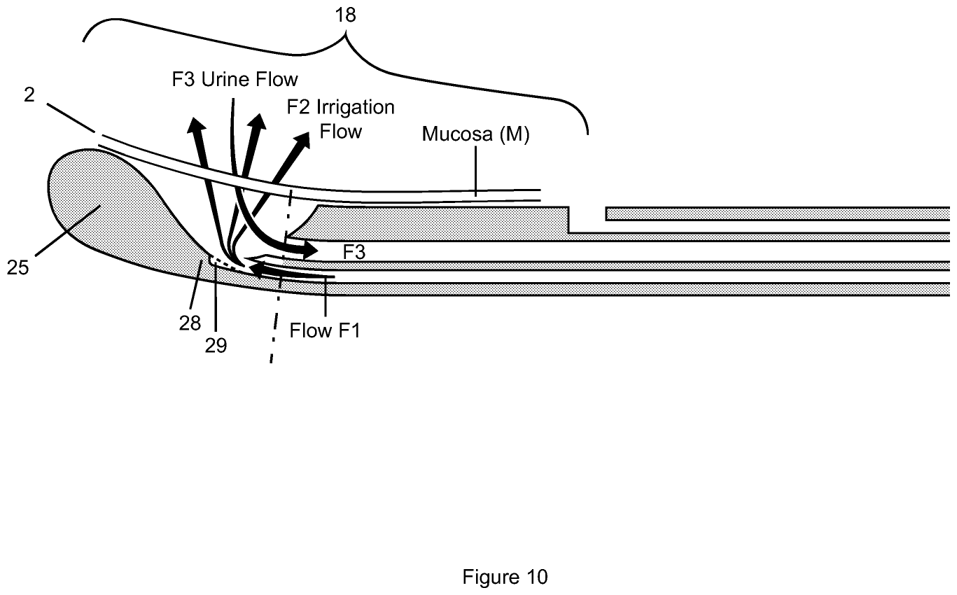

As shown the generally concave profile of this region intersects with the cylindrical body, the end portion of the catheter and the base to define an ‘open’ or ‘bowl’ region through which fluid may easily flow. Fluid flows in multiple directions as will be described with reference to FIGS. 9 and 10 below.

The cross-section shown in FIG. 7C shows how the side walls of the region incorporate 180-190 degrees of the circumference of the outside diameter (OD) of the tube. This thereby increases the area of the port compared to the ports of existing catheters. The design thus provides long, widened, smooth and curved edged ports with no ‘dead space’ distal to the drainage port to trap mucus or blood clots in the case of urethral catheters and surgical drains and curdled diet in the case of gastric decompression and naso-gastric and nasoi-intestinal enteral feeding tubes. As shown, the fluid ingress lumen is shown at the bottom of the region and within the base of the catheter tip.

FIG. 8 shows examples of the lumen areas of a catheter described herein. As illustrated the upper inflation lumen may be configured to communicate water as the inflation fluid. The central lumen communicates urine and the lower lumen communicates the irrigation fluid.

The relative areas in mm² are shown for each of 16, 18 and 20 Fr catheters. It will be recognised that these areas are only examples and that slight deviations from these parameters may also function.

The fluid flows associated with the catheter will now be described with reference to FIGS. 9 and 10.

FIG. 9 shows a closer cross-sectional view of the tip 18.

The tip 18 is formed of 3 distinct portions or regions. Specifically, the tip 18 comprises:
(i) a first portion 24 integral with the elongate generally cylindrical body 16 i.e. it is part of the cylindrical body of the catheter,
(ii) a second portion 25 distal to the first portion 24 and defining the distal end of the catheter 2; and
(iii) an intermediate fluid ingress/egress portion or region 26 located between the first and second portions.

Referring back to FIG. 7, the distance between the most distal end of the catheter and the centre of the inflation port is approximately 4 cm and the distance between the uppermost extent of the distal portion 27 and the intersection of the recess and the catheter body (shown as reference 26 in FIG. 9 is between 1.1 and 1.4 cm. These values have been identified as particularly advantageous geometries.

As discussed above with reference to FIG. 6 (a venous application of the catheter geometry) and FIGS. 7 (a urethral application of the catheter) the intermediate fluid ingress/egress portion is in the form of a generally concave recess extending from one side 16 of the tip (FIG. 7) towards an opposing side of the tip forming a base 28.

In effect, the recess defines an upper opening intersecting with the lumens 19 and 21 and thus allowing fluid communication into and out of the region.

Returning to FIG. 9, as shown, the lumen 21 extends through the base 28 to an approximate mid-point across region 26 where it terminates with a generally vertical or perpendicular opening allowing for the communication of fluid directly upwards. The importance of this flow path is described below.

As shown in FIG. 9 the base 28 mechanically couples the tip portion 24 and tip portion 25 together. As also shown in FIG. 7C the base only extends around a proportion of the circumference of the tip/elongate body. Specifically, the open angle defined by the recess accounts for between 170 and 190 degrees of the circumference of the tip/elongate body. The inventors have established that this angular range provides sufficient fluid passage through the sides of the intermediate region whilst providing sufficient mechanical strength to support the distal tip end as it is positioned in the bladder.

Returning to FIG. 9, F₃ depicts the flow of urine from the surrounding bladder into the recess of the long widened smooth and curved region 26 and exits into lumen 19 of the catheter to be connected into a urine bag at the proximal end of the catheter.

FIG. 9 also shows the flow of the irrigation fluid through the catheter and into the bladder. As shown, irrigation fluid flows along lumen 21 and through the base 28 to reach the lumen outlet 29. The lumen outlet 29 is configured to cause the fluid to change direction so as to be directed away from the base and outwards towards region 26. This is shown by arrow F₁. The region 26 is an open region into which urine may pass either from the top or from the sides of the catheter. The irrigation fluid flows away from the base 28 and towards the open upper boundary of the fluid ingress/egress region. This is show by arrows F₂.

The outlet 29 may also comprise a recess as illustrated by the dashed line at outlet 29 in FIG. 9. Such a recess into the catheter material creates a generally vertically or angled surface against which the irrigation fluid impinges as it leaves the outlet 29. Impinging on this surface may advantageously further cause an upward or generally vertical flow of irrigation fluid from the outlet 29 towards the mucosa thereby further improving the hydraulic effect of the irrigation fluid on the mucosa.

Furthermore, the upper extend i.e. the most distal end of the upper part of the outlet may be curved and may generally follow the profile of the catheter in an upwards direction and terminate at the intersection of the outlet 29 and recess 26 (illustrated by the sharp and curved profile proximal to the arrow F₁ in FIG. 9). In an alternative, and as also illustrated in FIG. 9, the outlet may terminate in a generally straight or vertical line as illustrated by the dashed and dotted line intersecting arrows F₁ and F₃ in FIG. 9.

The irrigation fluid serves a number of purposes as described in further detail with reference to FIG. 10.

The primary function of the ingress fluid through the catheter and to the catheter outlet port 29 is to provide a flow which applies an outward pressure against a mucosa or blood clots dietary contents or bladder lining or stomach lining that might otherwise obscure the fluid ingress/egress region. As shown in FIG. 10, the flow paths F₂ are such that they leave the outlet and flow upwards towards any obstruction or blockage. The fluid flow pushes the obstruction or blockage away from the fluid ingress/egress region to maintain the unobstructed volume immediately upstream of the outlet lumen 19. Maintaining the free flow of fluid into the region allows urine (in this example) to flow from around the catheter tip along flow line F₃ towards the outlet lumen and along lumen 19.

As shown in FIG. 10, the catheter is in abutment with the mucosa M of the bladder. As shown, by virtue of the side passages formed by the generally concave shape of recess 14, even when the mucosa 12 comes into contact with the catheter 2 a continuous passage for fluid flow is maintained by virtue of the open side passages defined by the opening in conjunction with the ingress irrigation fluid flow path. This arrangement prevents clogging by mucosa of the bladder in the case of urethral catheters, the mucosa of the stomach and small intestine in the case of gastric decompression and enteral feeding tubes.

The new catheter geometries provide a number of advantages including reduction in the incidence of uretheral catheter blockage, improved bladder drainage, reduced incidence of urinary infection and to reduced incidence of clot retention following urogenital tract surgery.

There may occasionally, despite the novel design of the drainage port, be a tendency of the mucosa to abut with the catheter and prolapse down within the cavity of the drainage port 26 blocking the flow of urine 19. However, the continuous irrigation flow F₂ will force the mucosa (M) out of the orifice of the drainage port 26 thereby maintaining an open and continuous flow path to the outlet lumen 19 as illustrated by flow F₃.

In effect the geometry of the tip of the catheter described herein functions in 2 complementary ways:
(i) First, as described above, the irrigation fluid is controlled and specifically directed to hydraulically push the mucosa away from the important exit lumen. This maintains a continuous flow until the bladder is fully empty.
(ii) Additionally, the geometry of the tip mechanically prevents the mucosa from encroaching and blocking the outlet lumen. By virtue of the perimeter of the recess described above and the intersection with the cylindrical elongate body 2 opposing mucosa supporting surfaces are provided—denoted by M₁ and M₂ in FIG. 9.

These two supporting surfaces or portion cooperate with the fluid flow and the angle of opening of the intermediate opening to allow for continuous flow through the catheter; something that is not possible with existing catheter designs.

Two additional features of the new geometry have been established by the applicant and are illustrated in FIG. 10. First, the end of the tip is provided with a smooth outer surface, much like a bullet shape. This eases the passage of the device through the urethra.

Furthermore, the bullet tip and aspiration port may advantageously be angled upwards of the main body of the catheter (as shown in FIG. 10).

This angle A may be between 16 degrees and 30 degrees, but is advantageously 30 degrees.

This is in order to facilitate passage of the catheter through the ‘bed’ of the prostate and neck of the bladder. From the manufacturing standpoint, the component constituting the bullet tip and aspiration port is likely to be required to be moulded onto the main body of the tubing—the site of the moulding is where the acute angle (allowing upwards project of the tip of the catheter) will be formed.

The inventions described herein are also applicable to other medical devices such as surgical drains as well as gastric decompression tubes and nasogastric and naso enteral feeding tubes.

Also, the improved design of the aspiration port will enhance the efficiency of drainage thereby reducing bladder sump volume and reducing the incidence of catheter-related urinary infection. As with the nasogastric feeding tube, blockage by bladder mucosal adherence and mucous produced from the wall of an inflamed bladder will be prevented. The ability to be able to apply internal coating to the polyurethane tubing will enable us to apply a bacteriostatic polymer) that will further reduce the incidence of catheter-related urinary infections. Not only will the invention benefit patients undergoing short-term urethral catheterisation but also those with long-term urinary catheterisation. The application of the port design will improve the functioning of venous catheters by virtue of improved flued patterns that in turn will reduce trauma during fluid infusion and thereby the incidence of thrombophlebitis and venous thrombosis that lead to the need for venous catheter withdrawal and replacement.

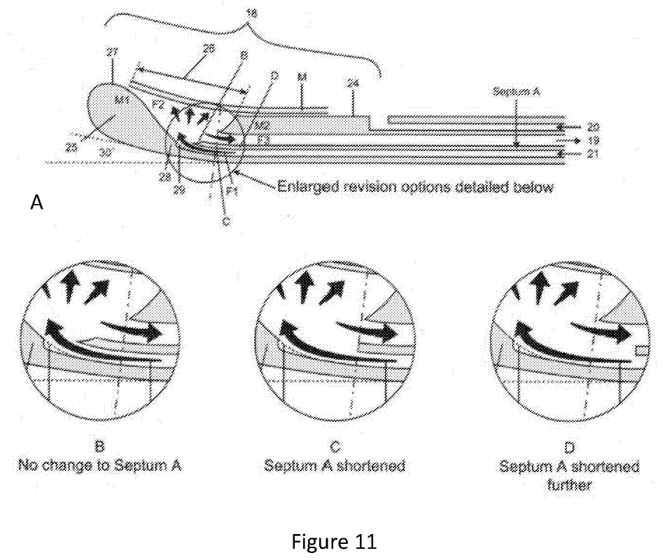

FIGS. 11A to 11D illustrate different positions of the septum with respect to the zone where the ingress and egress fluids mix. Specifically, FIG. 11A shows the un-truncated or shortened septum which directs ingress fluid to mix with urine before egressing from the catheter.

FIGS. 11B, 11C and 11D illustrate embodiments in which the septum may be shortened by varying amounts. In each embodiment the mixing and ingress fluid and urine will differ and allow for controllability of the fluid flows in the mixing zones. Thus, different catheter can be realised with different fluid mechanic properties.

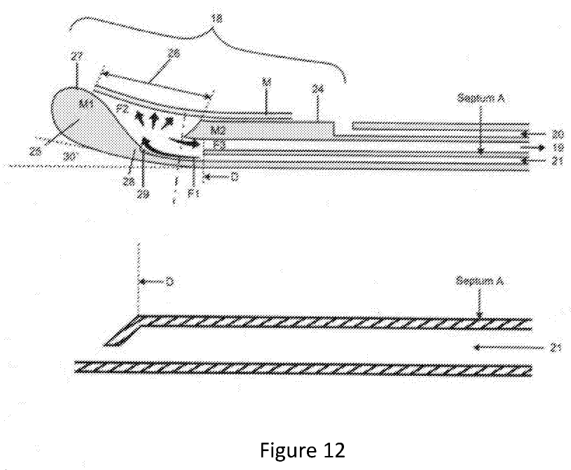

FIG. 12 illustrates a further embodiment of the distal end of the septum with a fluid directing projections. Specifically a projection is provided which causes a downward direction of flow of ingress fluid along the septum.

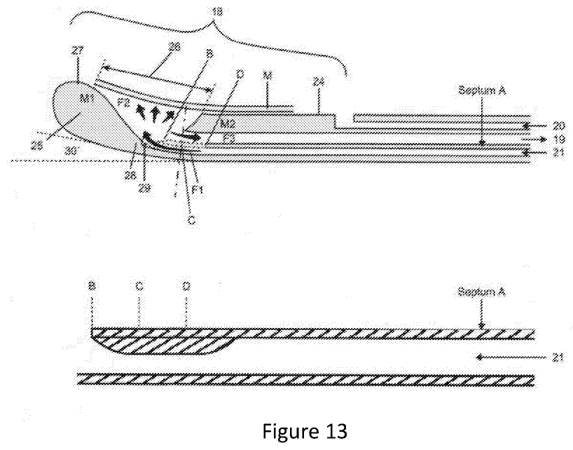

FIG. 13 illustrates a still further embodiment of the distal end of the septum with a modified fluid path projection. Here the fluid direction is achieved by providing an increased thickness to the upper surface of the septum. This causes a downward flow direction of ingress fluid.

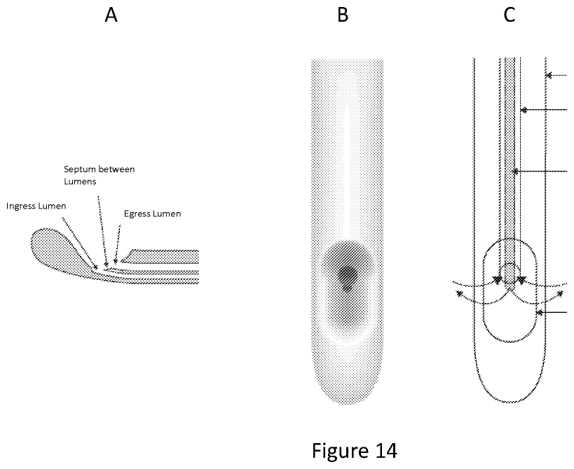

FIGS. 14A to 14C illustrate a plan view of the ingress and egress lumens and illustrates the intersection of ingress and egress ports. More specifically, FIGS. 14A to 14C schematically show the base of the exit port of the catheter. It will be noted how the opening of the egress channel lies above the opening of the ingress channel. This unique design feature allows the ingress irrigation fluid to enter into the exit port and MIX with the urine flow and dislodge impacted plugs of mucus or clots of blood into the lumen of the bladder.

The reference numerals for FIGS. 14A to 14C are as follows:
30 Urethral Catheter
31 Egress Lumen for Urine Flow
32 Ingress Lumen for Irrigation Fluid Flow
33 Flow of Urine and Irrigation Fluid
34 Exit Port
35 Opposing flow of Urine and Irrigation Fluid

1. A catheter comprising an elongate generally cylindrical body and a catheter tip for insertion into a human or animal, the elongate generally cylindrical body comprising a first lumen for fluid egress and two radially opposing lumens, a first being a fluid ingress lumen and a second being in fluid communication with an expandable member;
wherein the fluid ingress lumen is for communicating fluid along an axial direction of the elongate generally cylindrical body to the catheter tip and wherein the fluid egress lumen is for communicating fluid along the axial direction of the elongate generally cylindrical body from the catheter tip,
wherein the catheter tip comprises a first portion integral with the elongate generally cylindrical body, a second portion distal to the first portion and defining the distal end of the catheter and an intermediate fluid ingress/egress region located between the first and second portions,
wherein the intermediate fluid ingress/egress region is in the form of an open section of the catheter tip exposing the fluid ingress/egress lumens to allow for fluid communication; the catheter tip further comprising a base portion extending between the first and second catheter tip portions; and
wherein the fluid ingress lumen comprises an outlet port in fluid communication with the intermediate fluid ingress/egress region and wherein the fluid egress lumen comprises an opening in fluid communication with the intermediate fluid ingress/egress region, wherein the outlet port and the opening are oriented at an angle to a radial direction of the elongate generally cylindrical body, wherein the orientation of the outlet port and the opening is configured to cause mixing of the fluid specifically in the intermediate fluid ingress/egress region.

2. A catheter as claimed in claim 1, wherein the intermediate fluid ingress/egress region is in the form of a generally concave recess in the cylindrical body extending towards the base, the recess defining an upper opening relative to the base which intersects with the lumens to allow fluid flow to and from the lumens.

3. A catheter as claimed in claim 2, wherein the recess intersects with the base and first and second catheter tip portions to define a smooth and continuous surface profile.

4. A catheter as claimed in claim 1, wherein at least a portion of the intermediate fluid ingress/egress region extends 180 degrees or more around the circumference of the cylindrical body.

5. (canceled)

6. A catheter as claimed in claim 1, wherein the fluid egress lumen is coaxial with the elongate generally cylindrical body and intersects with the intermediate fluid ingress/egress region to allow fluid flow therefrom.

7. A catheter as claimed in claim 1, wherein the fluid ingress lumen extends along the elongate generally cylindrical body and intersects with the intermediate fluid ingress/egress region from the base portion to allow fluid flow substantially perpendicularly therefrom.

8. A catheter as claimed in claim 7, wherein fluid is arranged to flow from an outlet of the fluid ingress lumen so as to flow away from the inlet to the egress lumen.

9. A catheter as claimed in claim 1 wherein when viewed in cross-section the intermediate fluid ingress/egress region has a smooth generally U shaped profile.

10. A catheter as claimed in claim 1 wherein the second portion of the catheter tip defining the distal end of the catheter has a smooth generally curved outer surface.

11. A catheter as claimed in claim 1 wherein the second portion of the catheter tip defining the distal end of the catheter is inclined with respect to the elongate axis of the catheter.

12. A catheter as claimed in claim 11, wherein the second portion is inclined at an angle of between 15 degrees and 30 degrees

13. (canceled)

14. A catheter as claimed in claim 1 comprising the expandable member located circumferentially with respect to the elongate generally cylindrical body and movable radially outwards from the body surface.

15. A catheter as claimed in 14, wherein the expandable member is in the form of a circumferentially located balloon expandable between a first position in alignment with the outer surface of the elongate body and a second position radially spaced from the body.

16. A catheter as claimed in claim 14, wherein the lumen in fluid communication with the expandable member is configured to cause selective expansion or retraction of the expandable member.

17. (canceled)

18. A catheter as claimed in claim 14, wherein the expandable member is in the form of a balloon which may be selectively expanded by means of a fluid.

19. A urethral catheter as claimed in claim 14, wherein the expandable member is positioned at least 4 cm from the tip of the catheter to the midpoint of the expandable member inflation channel.

20. (canceled)

21. (canceled)

22. A catheter as claimed in claim 1 wherein the maximum length of the intermediate fluid ingress/egress region extending between the first portion of the catheter tip and the second portion of the catheter tip when measured along the axis of the catheter is less than 15 mm.

23. A catheter as claimed in claim 1 wherein the catheter is a urethral catheter or surgical drainage catheter.

24. A urethral catheter comprising an elongate generally cylindrical body and a catheter tip for insertion into and along a urethra,
the elongate generally cylindrical body comprising:
an irrigation lumen for communicating fluid to the catheter tip,
a drainage lumen for receiving fluid from the catheter tip, and
an inflation lumen in fluid communication with an expandable member;
wherein the catheter tip has a first and second mucosa supporting portion and an intermediate opening between the first and second mucosa supporting portions, wherein the opening extends between 180 degrees and 190 degrees around the circumference of the elongate generally cylindrical body, the intermediate opening being in fluid communication with the irrigation lumen and the drainage lumen.

25. A method of urethral or surgical drainage or gastric decompression or enteral feeding tube for use in the stomach and small intestine by means of a catheter, the catheter comprising:
an elongate generally cylindrical body and a catheter tip for insertion into a human or animal, the elongate generally cylindrical body comprising a first lumen for fluid egress and two radially opposing lumens, a first being a fluid ingress lumen and a second being in fluid communication with an expandable member;
wherein the fluid ingress lumen is for communicating fluid along an axial direction of the elongate generally cylindrical body to the catheter tip and wherein the fluid egress lumen is for communicating fluid along the axial direction of the elongate generally cylindrical body from the catheter tip,
wherein the catheter tip comprises a first portion integral with the elongate generally cylindrical body, a second portion distal to the first portion and defining the distal end of the catheter and an intermediate fluid ingress/egress region located between the first and second portions,
wherein the intermediate fluid ingress/egress region is in the form of an open section of the catheter tip exposing the fluid ingress/egress lumens to allow for fluid communication; the catheter tip further comprising a base portion extending between the first and second catheter tip portions; and
wherein the fluid ingress lumen comprises an outlet port in fluid communication with the intermediate fluid ingress/egress region and wherein the fluid egress lumen comprises an opening in fluid communication with the intermediate fluid ingress/egress region, wherein the outlet port and the opening are oriented at an angle to a radial direction of the elongate generally cylindrical body, wherein the orientation of the outlet port and the opening is configured to cause mixing of the fluid specifically in the intermediate fluid ingress/egress region, the method comprising the steps of:
inserting the catheter into a body, causing fluid to ingress into a body and removing fluid from the body through the fluid egress lumen.
